(19) United States
(12) Patent Application Publication
(10) Pub. No.: US 2026/0097238 A1
(43) Pub. Date: Apr. 9, 2026

(54) TREATMENT FACILITY AND RADIATION TREATMENT APPARATUS

(71) Applicant: SUMITOMO HEAVY INDUSTRIES, LTD., Tokyo (JP)

(72) Inventor: Masaru NAKAKITA, Niihama-shi (JP)

(21) Appl. No.: 19/342,413

(22) Filed: Sep. 26, 2025

(30) Foreign Application Priority Data
Oct. 8, 2024 (JP) 2024-176580

(51) Int. Cl. A61N 5/10

(52) U.S. Cl. CPC A61N 5/1048; A61N 2005/109; A61N 2005/1097

(57) ABSTRACT

A treatment facility that irradiates an irradiation target with radiation to perform treatment includes: an irradiation unit that emits the radiation; a wall portion in which an irradiation port of the irradiation unit is provided; and a movable door that is provided to face the wall portion so as to cover the irradiation port, in which the movable door slides along the wall portion to both sides of the irradiation port in a lateral direction.

### CROSS-REFERENCE TO RELATED APPLICATIONS

This application claims priority to Japanese Patent Application No. 2024-176580, filed on Oct. 8, 2024, which is incorporated by reference herein in its entirety.

### BACKGROUND

### Technical Field

Certain embodiments of the present invention relate to a treatment facility and a radiation treatment apparatus.

### Description of Related Art

Boron neutron capture therapy (BNCT) using a boron compound is known as a neutron capture therapy that emits a neutron ray to kill cancer cells. In the boron neutron capture therapy, boron that has been incorporated into the cancer cells in advance is irradiated with the neutron ray, and the resulting scattering of heavy charged particles selectively destroys the cancer cells.

For example, a radiation treatment apparatus disclosed in the related art is known as the above-described radiation treatment apparatus. The radiation treatment apparatus disclosed in the related art performs treatment by disposing a patient, who is fixed to a mounting member, in front of an irradiation port for neutron rays. Therefore, the patient is positioned in a preparation room. Then, the patient is transported to a treatment room and is positioned again.

### SUMMARY

According to an embodiment of the present invention, there is provided a treatment facility that irradiates an irradiation target with radiation to perform treatment. The treatment facility includes: an irradiation unit that emits the radiation; a wall portion in which an irradiation port of the irradiation unit is provided; and a movable door that is provided to face the wall portion so as to cover the irradiation port. The movable door slides along the wall portion to both sides of the irradiation port in a lateral direction.

According to another embodiment of the present invention, there is provided a radiation treatment apparatus that irradiates an irradiation target with radiation to perform treatment. The radiation treatment apparatus includes: an irradiation unit that emits the radiation; and a movable door that is provided to face a wall portion, in which an irradiation port of the irradiation unit is provided, so as to cover the irradiation port. The movable door slides along the wall portion to both sides of the irradiation port in a lateral direction.

### BRIEF DESCRIPTION OF THE DRAWINGS

FIG. 1 is a schematic view showing a treatment facility and a radiation treatment apparatus according to an embodiment of the present invention.

FIG. 2 is a schematic view showing the treatment facility and the radiation treatment apparatus according to the embodiment of the present invention.

FIG. 3 is a front view showing a structure of a movable door.

FIG. 4 is a side view showing the structure of the movable door.

FIGS. 5A and 5B are schematic views showing a sliding aspect of the movable door.

FIG. 6 is a view showing a positional relationship between a detection unit and the movable door.

### DETAILED DESCRIPTION

In the above-described radiation treatment apparatus, in some cases, a movable door is provided to cover the irradiation port in order to suppress leakage of the radiation from the irradiation port for radiation. This movable door is divided into a pair of door members and has a mechanism in which the door members slide open simultaneously to the left and right sides of the irradiation port, respectively. Therefore, when treatment is performed, the movable door is opened. When treatment is not performed, the movable door is closed. Here, in the radiation treatment apparatus, in some cases, a treatment table on which the patient is fixed is disposed in various directions and orientations according to the content of the treatment. In this case, a movement range of the treatment table may be restricted such that the treatment table and an open flat plate do not interfere with each other. Alternatively, another issue is that the space of the treatment room needs to increase in order to secure a space for opening the movable door so as not to interfere with the treatment table.

Therefore, it is desirable to provide a treatment facility and a radiation treatment apparatus that can reduce constraints on a movement range of a treatment table while suppressing an increase in the size of the treatment room.

The treatment facility according to the embodiment of the present invention includes the wall portion in which the irradiation port of the irradiation unit is provided and the movable door that is provided to face the wall portion so as to cover the irradiation port. Therefore, when treatment is not performed, the movable door covers the irradiation port, which makes it possible to suppress the leakage of the radiation into the treatment room. The movable door slides along the wall portion to both sides of the irradiation port in the lateral direction. Therefore, during treatment, the movable door slides such that the radiation can be emitted from the irradiation port. That is, the movable door is different from the structure in which a pair of door members slide open simultaneously to the left and right sides, respectively, in that, when the movable door slides to one side in the lateral direction, the movable door is not present on the other side and, when the movable door slides to the other side in the lateral direction, the movable door is not present on the one side. Therefore, when the treatment table moves to be close to the wall portion, the movable door slides to the side opposite to the treatment table, which makes it possible to secure a space for the treatment table. In this case, it is possible to suppress the interference of the movable door with the treatment table without the movable door being separated from the irradiation port more than necessary. Therefore, the treatment room may not need to be enlarged more than necessary in order to secure a space for the movable door after the sliding. From the above, it is possible to reduce constraints on a movement range of the treatment table while suppressing an increase in the size of the treatment room.

During a first sliding operation, the movable door may slide to one side in the lateral direction with respect to the irradiation port. During a second sliding operation, the movable door may slide to the other side in the lateral direction with respect to the irradiation port. This makes it possible for the movable door to be in a state in which the movable door is not present on the other side during the first sliding operation and is not present on the one side during the second sliding operation.

The treatment facility may further include: a guide unit that is provided on one of an upper end side and a lower end side of the movable door to guide the sliding of the movable door in the lateral direction; and a restricting unit that is provided on the other of the upper end side and the lower end side of the movable door to restrict movement of the movable door in a thickness direction. This makes it possible for the movable door to smoothly slide without rattling.

The treatment facility may further include: a treatment table on which the irradiation target is placed; and a control unit that performs calculations related to the treatment. The control unit may calculate a direction and a position of the treatment table based on a treatment plan to determine a sliding direction of the movable door. This makes it possible for the control unit to determine the operations of the treatment table and the movable door based on the treatment plan.

The control unit may control the movable door such that the movable door slides in the determined sliding direction. In this case, it is possible to automatically control the movable door.

The treatment facility may further include a detection unit that detects a position of the movable door, and the detection unit may be provided on a ceiling side. This disposition of the detection unit at a high position makes it possible to suppress the erroneous detection of a worker or another member in the treatment room by the detection unit.

The radiation may be a neutron ray. In this case, it is possible to obtain the above-described effects when treatment is performed by the neutron capture therapy in which the movement range of the treatment table is wide.

According to the radiation treatment apparatus, it is possible to obtain the same operations and effects as those of the above-described treatment facility.

Hereinafter, a preferred embodiment of the present invention will be described in detail with reference to the drawings.

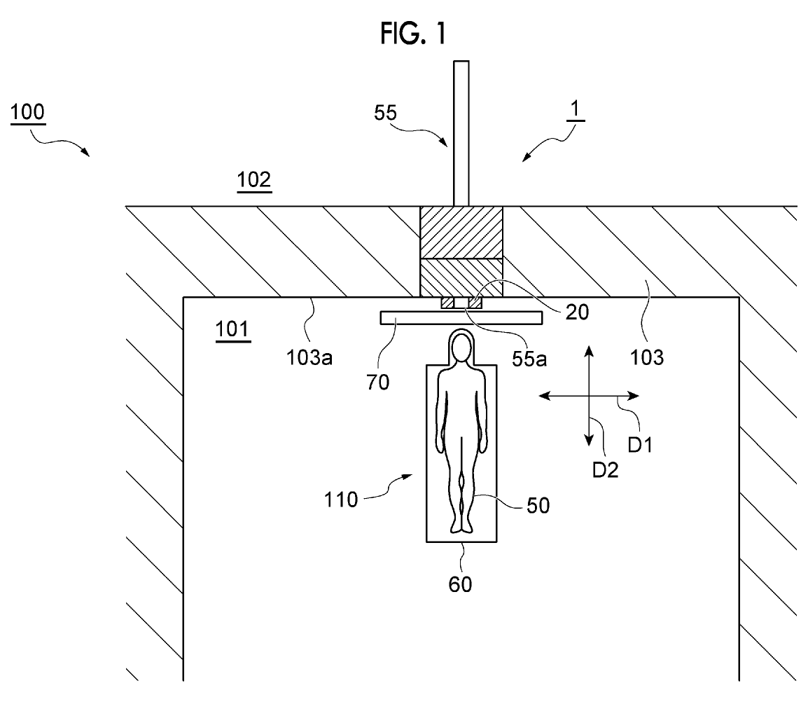

FIG. 1 is a schematic view showing a treatment facility 100 and a radiation treatment apparatus 1 according to an embodiment of the present invention. In the present embodiment, a neutron capture therapy apparatus that performs cancer treatment using boron neutron capture therapy (BNCT) is given as an example of the radiation treatment apparatus 1. In addition, the radiation treatment apparatus 1 is not limited to the neutron capture therapy apparatus, and a radiation treatment apparatus using X-rays, proton beams, heavy particle beams, or the like may be adopted.

As shown in FIG. 1, the treatment facility 100 includes the radiation treatment apparatus 1, a treatment room 101 in which a patient 50 (irradiation target) is treated by the radiation treatment apparatus 1, and an accelerator room 102 in which an accelerator 2, which will be described below, is disposed. In addition, the treatment facility 100 includes a wall portion 103 that separates the treatment room 101 from the accelerator room 102.

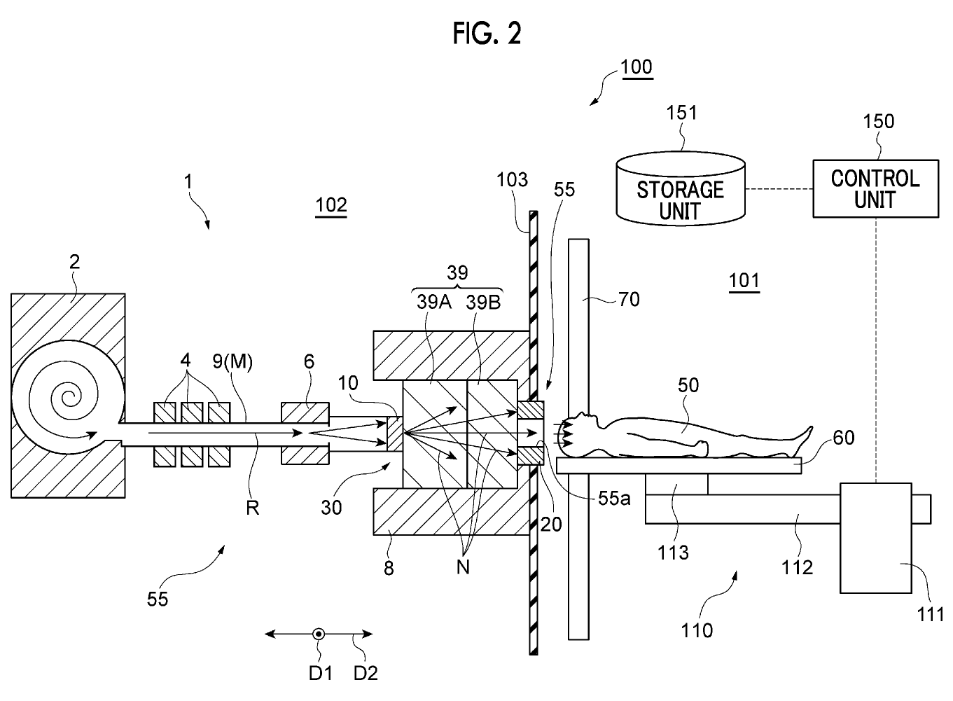

The radiation treatment apparatus 1 will be described with reference to FIG. 2. The radiation treatment apparatus 1 irradiates a tumor in the patient 50, who has been administered, for example, boron (¹⁰B), with neutron rays N. The radiation treatment apparatus 1 includes the accelerator 2. The accelerator 2 accelerates particles to emit a particle beam R. For example, a cyclotron, a linear accelerator, or the like may be adopted as the accelerator 2.

The particle beam R emitted from the accelerator 2 passes through a transport path 9 called a beam duct, of which the inside is maintained in a vacuum and through which the beam can pass, and is transported to a target disposition portion 30. The target disposition portion 30 is a portion in which a target 10 is disposed and has a mechanism that holds the target 10 in a posture for irradiation. In the target disposition portion 30, the target 10 is disposed at a position facing an end portion of the transport path 9. The particle beam R emitted from the accelerator 2 passes through the transport path 9 and advances toward the target 10 disposed in the end portion of the transport path 9. A plurality of electromagnets 4 (quadrupole electromagnets or the like) and a scanning electromagnet 6 are provided along the transport path 9. The plurality of electromagnets 4 adjust, for example, an axis of the particle beam R using the electromagnets.

The scanning electromagnet 6 is used to perform scanning with the particle beam R and to control the irradiation of the target 10 with the particle beam R. The scanning electromagnet 6 controls a position where the target 10 is irradiated with the particle beam R.

The radiation treatment apparatus 1 irradiates the target 10 with the particle beam R to generate the neutron ray N and emits the neutron ray N toward the patient 50. The radiation treatment apparatus 1 includes the target 10, a shield member 8, a deceleration member 39, and a collimator 20.

The target 10 is irradiated with the particle beam R to generate the neutron ray N. The target 10 is a solid member made of a material that is irradiated with the particle beam R to generate the neutron ray N. Specifically, the target 10 is made of, for example, beryllium (Be), lithium (Li), tantalum (Ta), or tungsten (W) and has, for example, a solid disk shape with a diameter of 160 mm. Further, the target 10 is not limited to the disk shape and may have another shape.

The deceleration member 39 decelerates the neutron ray N generated by the target 10 (reduces energy of the neutron ray N). The deceleration member 39 may have a stacked structure of a layer 39A that mainly decelerates fast neutrons included in the neutron ray N and a layer 39B that mainly decelerates epithermal neutrons included in the neutron ray N.

The shield member 8 shields the generated neutron ray N and gamma rays or the like generated with the generation of the neutron ray N so as not to be emitted to the outside. The shield member 8 is provided to surround the deceleration member 39. Upper and lower portions of the shield member 8 extend from the deceleration member 39 to an upstream side of the particle beam R.

The collimator 20 shapes an irradiation field of the neutron ray N and includes an irradiation port 55a through which the neutron ray N passes. The collimator 20 is, for example, a block-shaped member having an irradiation port at the center. The collimator 20 is attached to a location of the wall portion 103 where the neutron ray N is emitted into the treatment room 101.

The accelerator 2, the transport path 9, the target 10, the deceleration member 39, and the collimator 20 constitute an irradiation unit 55 that performs irradiation with the neutron ray. The irradiation port of the collimator 20 corresponds to the irradiation port 55a of the irradiation unit 55. The irradiation port 55a of the irradiation unit 55 is provided in the wall portion 103. In addition, the collimator 20 may not be provided in the wall portion 103 and may be provided in a treatment table 60. A horizontal direction along the wall portion 103 is defined as a lateral direction D1. A direction in which an irradiation axis of the neutron ray N emitted from the irradiation port 55a extends is defined as a front-rear direction D2.

The radiation treatment apparatus 1 has a moving mechanism 110. The moving mechanism 110 has a base portion 111 that is provided on a floor surface at a position separated from the irradiation port 55a by a predetermined distance. The base portion 111 is a member that extends in an up-down direction, can be rotated about an axis extending in the up-down direction, and can be moved up and down in the up-down direction. The base portion 111 has an arm portion 112 that extends in the horizontal direction. A support portion 113 that supports the treatment table 60 is provided at a tip part of the arm portion 112. The arm portion 112 can be expanded and contracted to change the position of the support portion 113. In addition, the arm portion 112 can adjust the inclination of the support portion 113. The support portion 113 can be rotated about an axis that extends in the up-down direction at the tip part of the arm portion 112.

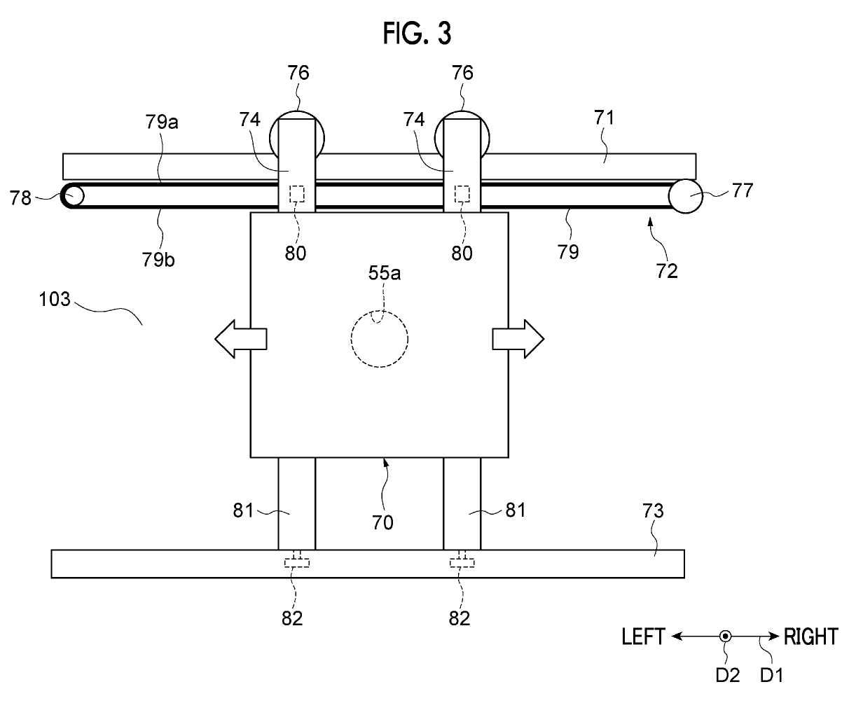

The radiation treatment apparatus 1 includes a movable door 70. The movable door 70 is provided to face the wall portion 103 so as to cover the irradiation port 55a. The movable door 70 is disposed between the wall portion 103 and the moving mechanism 110. The movable door 70 is provided to extend in the up-down direction and the lateral direction D1 so as to face the wall portion 103. A thickness direction of the movable door 70 is disposed to coincide with the front-rear direction D2. When viewed from the front, the movable door 70 is disposed to cover the entire irradiation port 55a (see FIG. 3). The movable door 70 has a rectangular shape (see FIG. 3). However, the shape of the movable door 70 is not limited thereto and may be appropriately changed. A gap between the movable door 70 and a surface 103a of the wall portion 103 is set to a size that can suppress the leakage of the radiation from the irradiation port 55a. Further, when the collimator 20 protrudes from the surface 103a of the wall portion 103, the movable door 70 is disposed so as not to interfere with a tip part of the collimator 20. A material forming the movable door 70 is not particularly limited as long as the material can shield the radiation, and the movable door 70 is made of a material such as lead, iron, or concrete.

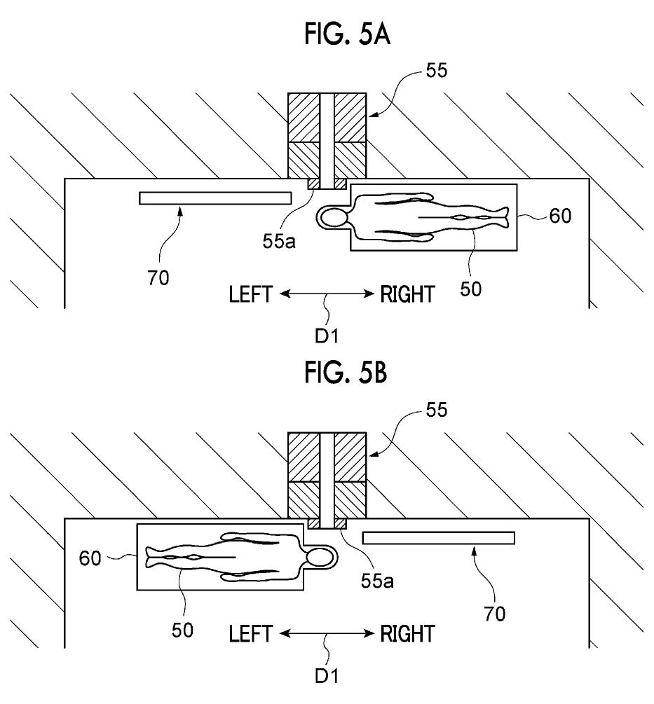

The movable door 70 will be described in detail with reference to FIGS. 3 to 5B. In addition, in the following description, "left" and "right" are based on the posture when viewing the irradiation port 55a in the treatment room 101. As shown in FIG. 3 and FIGS. 5A and 5B, the movable door 70 slides along the wall portion 103 to both sides of the irradiation port 55a in the lateral direction D1. That is, the movable door 70 can slide to the left side in the lateral direction D1 with respect to the irradiation port 55a (see FIG. 5A). The movable door 70 can slide to the right side in the lateral direction D1 with respect to the irradiation port 55a (see FIG. 5B). The movable door 70 is formed as an indivisible unit, unlike a door in which a pair of door members slide open simultaneously to the left and right sides, respectively. During a first sliding operation, the movable door 70 slides to the left side in the lateral direction D1 with respect to the irradiation port 55a. During a second sliding operation, the movable door 70 slides to the right side in the lateral direction D1 with respect to the irradiation port 55a. Therefore, when the movable door 70 slides to the left side of the irradiation port 55a, the entire movable door 70 slides to the left side, and the movable door 70 is not present on the right side of the irradiation port 55a. When the movable door 70 slides to the right side of the irradiation port 55a, the entire movable door 70 slides to the right side, and the movable door 70 is not present on the left side of the irradiation port 55a.

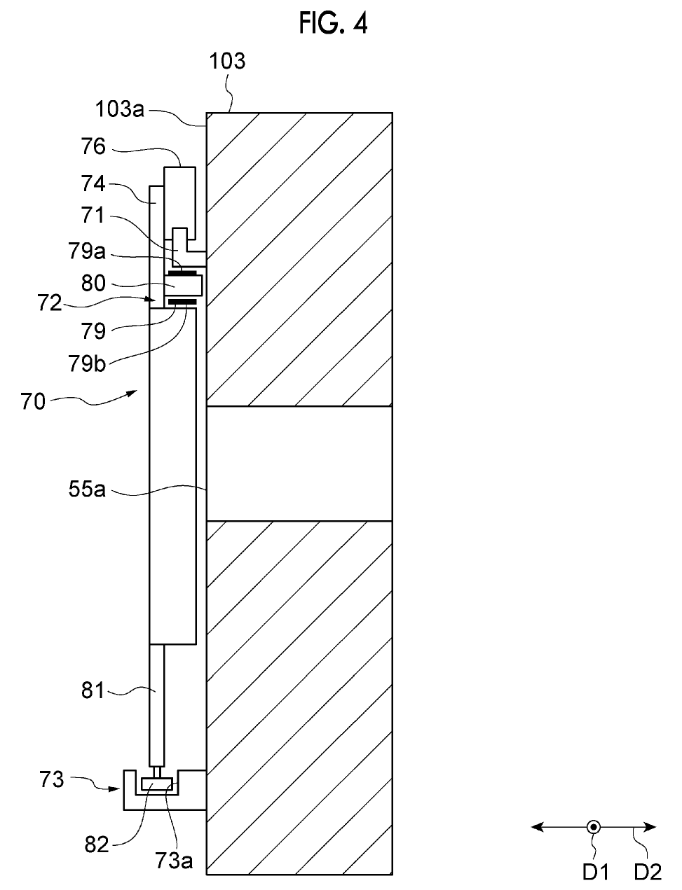

As shown in FIGS. 3 and 4, the radiation treatment apparatus 1 includes a guide unit 71, a drive unit 72, and a restricting unit 73. The guide unit 71 and the drive unit 72 are provided on an upper end side of the movable door 70. The restricting unit 73 is provided on a lower end side of the movable door 70.

The guide unit 71 is a member that guides the sliding of the movable door 70 in the lateral direction D1. The guide unit 71 is provided in the wall portion 103 on the upper side of the movable door 70 to extend in the lateral direction D1. The movable door 70 includes a pair of pillar portions 74 that extend upward from an upper end portion and a pair of wheel portions 76 that are provided in the pillar portions 74, respectively. The pillar portion 74 rotatably supports the wheel portion 76. The wheel portion 76 is disposed such that a rotary shaft extends in the front-rear direction D2. An outer peripheral surface of the wheel portion 76 is placed on the upper end portion of the guide unit 71. Therefore, the movable door 70 slides in the lateral direction D1 by the traveling of the wheel portion 76 on the guide unit 71.

The drive unit 72 is a mechanism that applies a driving force for sliding to the movable door 70. The drive unit 72 is provided between the movable door 70 and the guide unit 71. The drive unit 72 includes a drive pulley 77, a driven pulley 78, a belt 79, and a connecting portion 80. The pulleys 77 and 78 are disposed such that rotary shafts thereof extend in the front-rear direction D2. The drive pulley 77 is disposed on one end side (right side) of the drive unit 72 in the lateral direction D1. The driven pulley 78 is disposed on the other end side (left side) of the drive unit 72 in the lateral direction D1. The drive pulley 77 is a pulley that is connected to a motor (not shown) and is rotated to apply a driving force to the belt 79. The belt 79 is an annular endless belt. The belt 79 is stretched over the drive pulley 77 and the driven pulley 78. The belt 79 has an upper portion 79a that is stretched over an upper end portion of the drive pulley 77 and an upper end portion of the driven pulley 78 and a lower portion 79b that is stretched over a lower end portion of the drive pulley 77 and a lower end portion of the driven pulley 78. The connecting portion 80 connects the pillar portion 74 and the upper portion 79a of the belt 79. Therefore, the drive pulley 77 is rotated to apply a driving force for sliding in the lateral direction D1 to the movable door 70 via the belt 79, the connecting portion 80, and the pillar portion 74.

The restricting unit 73 is a member that restricts the movement of the movable door 70 in the thickness direction, here, in the front-rear direction D2. The restricting unit 73 is provided in the wall portion 103 on the lower side of the movable door 70 so as to extend in the lateral direction D1. The restricting unit 73 has a groove portion 73a extending along the lateral direction D1 in an upper surface thereof. The movable door 70 includes a pair of pillar portions 81 that extend downward from the lower end portion and a pair of roller portions 82 that are provided in the pillar portions 81, respectively. The pillar portion 81 rotatably supports the roller portion 82. The roller portion 82 is disposed such that a rotary shaft extends in the up-down direction. The roller portion 82 is inserted into the groove portion 73a. Therefore, when the movable door 70 slides in the lateral direction D1, the roller portion 82 is guided by the groove portion 73a such that the movement of the movable door 70 in the front-rear direction D2 is restricted.

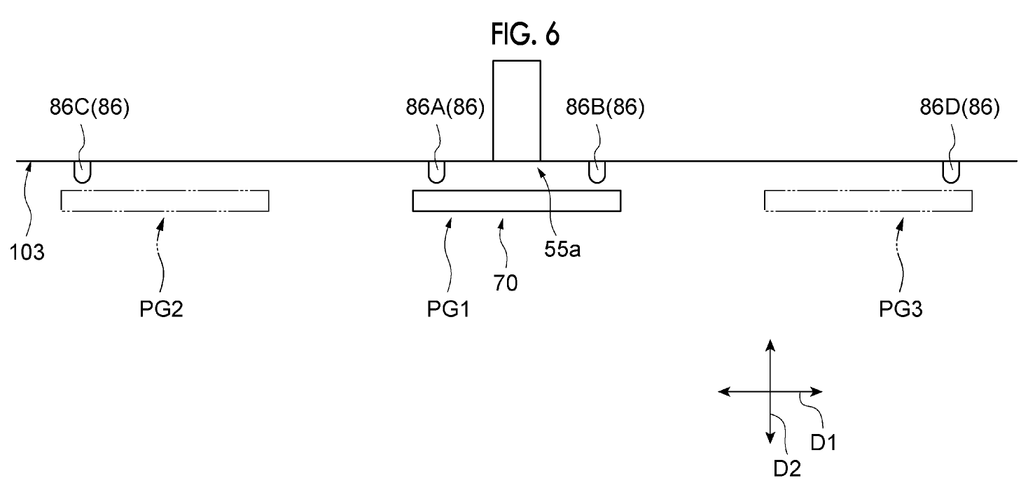

The radiation treatment apparatus 1 includes a detection unit 86 that detects the position of the movable door 70 as shown in FIG. 6. The detection unit 86 is provided on a ceiling side. That is, the detection unit 86 is provided at a position higher than the irradiation port 55a. In the example shown in FIG. 6, the radiation treatment apparatus 1 has detection units 86A, 86B, 86C, and 86D. The detection units 86A and 86B detect that the movable door 70 is disposed at a position PG1 for covering the irradiation port 55a. The detection units 86A and 86B are disposed on the left and right sides of the irradiation port 55a, respectively. The detection unit 86C detects that the movable door 70 is disposed at a left retracted position PG2. The detection unit 86C is disposed at a position that is spaced to the left side of the detection unit 86A. The detection unit 86D detects that the movable door 70 is disposed at a right retracted position PG3. The detection unit 86D is disposed at a position that is spaced to the right side of the detection unit 86B.

As shown in FIG. 2, the radiation treatment apparatus 1 includes a control unit 150 that controls various devices and a storage unit 151 that stores various types of information used for control. The control unit 150 controls the irradiation unit 55, the moving mechanism 110, and the movable door 70. The control unit 150 performs calculations related to the treatment to be performed by the radiation treatment apparatus 1. The storage unit 151 stores various types of data necessary for the control unit 150 to perform calculations related to treatment. The control unit 150 acquires data of a treatment plan that defines what kind of treatment the radiation treatment apparatus 1 will perform. The treatment plan includes the position of an affected part of the patient 50, the posture of the patient 50, and the dose of the neutron ray N with which the patient 50 is irradiated.

The control unit 150 calculates the direction and position of the treatment table 60 based on the treatment plan to determine the sliding direction of the movable door 70. For example, the control unit 150 can determine that the treatment table 60 is disposed in a direction in which the treatment table 60 extends in the front-rear direction D2 in front of the irradiation port 55a as shown in FIG. 1. In this case, the control unit 150 may set either the right side or the left side as the sliding direction of the movable door 70.

In addition, as shown in FIG. 5A, the control unit 150 can determine that the treatment table 60 is disposed in a direction in which a head-side end portion of the treatment table 60 is disposed in front of the irradiation port 55a and the treatment table 60 extends in the right direction. In this case, the control unit 150 determines the sliding direction of the movable door 70 to be the left side. In addition, as shown in FIG. 5B, the control unit 150 can determine that the treatment table 60 is disposed in a direction in which the head-side end portion of the treatment table 60 is disposed in front of the irradiation port 55a and the treatment table 60 extends in the left direction. In this case, the control unit 150 determines the sliding direction of the movable door 70 to be the right side.

The control unit 150 controls the movable door 70 such that the movable door 70 slides in the determined sliding direction. That is, when the movable door 70 is slid to the left side, the control unit 150 transmits a control signal to the drive unit 72 (see FIG. 3) to slide the movable door 70 to the left side. When the movable door 70 is detected by the left detection unit 86C, the control unit 150 stops the sliding. When the movable door 70 is slid to the right side, the control unit 150 transmits a control signal to the drive unit 72 (see FIG. 3) to slide the movable door 70 to the right side. When the movable door 70 is detected by the right detection unit 86D, the control unit 150 stops the sliding. The control unit 150 controls the moving mechanism 110 such that the treatment table 60 is disposed in the determined direction. In addition, after the treatment is completed, the control unit 150 returns the treatment table 60 to the original state and returns the movable door 70 to the position of the irradiation port 55a based on the detection results of the detection units 86A and 86B.

Next, the operations and effects of the treatment facility 100 and the radiation treatment apparatus 1 according to the present embodiment will be described.

The treatment facility 100 according to the present embodiment includes the wall portion 103 in which the irradiation port 55a of the irradiation unit 55 is provided and the movable door 70 that is provided to face the wall portion 103 so as to cover the irradiation port 55a. Therefore, when treatment is not performed, the movable door 70 covers the irradiation port 55a, which makes it possible to suppress the leakage of the radiation into the treatment room 101. The movable door 70 slides along the wall portion 103 to both sides of the irradiation port 55a in the lateral direction D1. Therefore, during treatment, the movable door 70 slides such that the radiation can be emitted from the irradiation port 55a. Here, as a comparative example, a structure is given in which a movable door has a pair of door members and the pair of door members slide open simultaneously to the left and right sides, respectively. In this structure according to the comparative example, the door member on one side in the lateral direction D1 slides to the one side in the lateral direction D1, and the door member on the other side in the lateral direction D1 slides to the other side in the lateral direction D1. As a result, the door members are present on both sides of the irradiation port 55a in the lateral direction D1, respectively. In contrast, in the treatment facility 100 according to the present embodiment, the movable door 70 is different from the structure in which the pair of door members slide open simultaneously to the left and right sides, respectively, in that, when the movable door 70 slides to one side in the lateral direction D1, the movable door 70 is not present on the other side and, when the movable door 70 slides to the other side in the lateral direction D1, the movable door 70 is not present on the one side. Therefore, when the treatment table 60 is moved to be close to the wall portion 103 (a state shown in FIGS. 5A and 5B), the movable door 70 slides to the side opposite to the treatment table 60, which makes it possible to secure a space for the treatment table 60. In this case, it is possible to suppress the interference of the movable door 70 with the treatment table 60 without the movable door 70 being separated from the irradiation port 55a more than necessary. Therefore, the treatment room 101 may not need to be enlarged more than necessary in order to secure a space for the movable door 70 after the sliding. From the above, it is possible to reduce constraints on a movement range of the treatment table 60 while suppressing an increase in the size of the treatment room 101.

In addition, during the first sliding operation, the movable door 70 slides to the left side (one side) in the lateral direction D1 with respect to the irradiation port 55a. During the second sliding operation, the movable door 70 slides to the right side (the other side) in the lateral direction D1 with respect to the irradiation port 55a. This makes it possible for the movable door 70 to be in a state in which the movable door 70 is not present on the other side during the first sliding operation and is not present on the one side during the second sliding operation.

The treatment facility 100 may further include the guide unit 71 that is provided on one of the upper end side and the lower end side of the movable door 70 to guide the sliding of the movable door 70 in the lateral direction D1 and the restricting unit 73 that is provided on the other of the upper end side and the lower end side of the movable door 70 to restrict the movement of the movable door 70 in the front-rear direction D2 (the thickness direction of the movable door). This makes it possible for the movable door 70 to slide smoothly without rattling.

The treatment facility 100 may further include the treatment table 60 on which an irradiation target is placed and the control unit 150 that performs calculations related to treatment. The control unit 150 may calculate the direction and position of the treatment table 60 based on the treatment plan to determine the sliding direction of the movable door 70. This makes it possible for the control unit 150 to determine the operations of the treatment table 60 and the movable door 70 based on the treatment plan.

The control unit 150 may control the movable door 70 such that the movable door 70 slides in the determined sliding direction. In this case, it is possible to automatically control the movable door 70.

The treatment facility 100 may further include the detection unit 86 that detects the position of the movable door 70, and the detection unit 86 may be provided on the ceiling side. This disposition of the detection unit 86 at a high position makes it possible to suppress the erroneous detection of a worker or another member in the treatment room 101 by the detection unit 86.

The radiation may be a neutron ray. In this case, it is possible to obtain the above-described effects when treatment is performed by the neutron capture therapy in which the movement range of the treatment table 60 is wide.

The radiation treatment apparatus 1 according to the present embodiment irradiates an irradiation target with radiation to perform treatment and includes the irradiation unit 55 that emits the radiation and the movable door 70 that is provided to face the wall portion 103, in which the irradiation port 55a of the irradiation unit 55 is provided, so as to cover the irradiation port 55a. The movable door 70 slides along the wall portion 103 to both sides of the irradiation port 55a in the lateral direction D1.

According to the radiation treatment apparatus 1, it is possible to obtain the same operation and effects as those of the treatment facility 100.

The present invention is not limited to the above-described embodiment.

For example, the structures of the treatment facility and the radiation treatment apparatus shown in FIGS. 1 and 2 are only examples and can be appropriately changed. In addition, the drive mechanism of the movable door 70 is not limited to that shown in FIGS. 3 and 4 and can be appropriately changed.

It should be understood that the invention is not limited to the above-described embodiment, but may be modified into various forms on the basis of the spirit of the invention. Additionally, the modifications are included in the scope of the invention.

What is claimed is:

1. A treatment facility that irradiates an irradiation target with radiation to perform treatment, the treatment facility comprising:
an irradiation unit that emits the radiation;
a wall portion in which an irradiation port of the irradiation unit is provided; and
a movable door that is provided to face the wall portion so as to cover the irradiation port,
wherein the movable door slides along the wall portion to both sides of the irradiation port in a lateral direction.

2. The treatment facility according to claim 1,
wherein, during a first sliding operation, the movable door slides to one side in the lateral direction with respect to the irradiation port, and
during a second sliding operation, the movable door slides to the other side in the lateral direction with respect to the irradiation port.

3. The treatment facility according to claim 1, further comprising:
a guide unit that is provided on one of an upper end side and a lower end side of the movable door to guide the sliding of the movable door in the lateral direction; and
a restricting unit that is provided on the other of the upper end side and the lower end side of the movable door to restrict movement of the movable door in a thickness direction.

4. The treatment facility according to claim 3, further comprising:
a drive unit that is provided between the movable door and the guide unit and applies a driving force for sliding to the movable door.

5. The treatment facility according to claim 4,
wherein the movable door includes a pair of pillar portions that extend downward from a lower end portion and a pair of roller portions that are provided in the pillar portions, respectively.

6. The treatment facility according to claim 5,
wherein the pillar portion rotatably supports the roller portion.

7. The treatment facility according to claim 6,
wherein the restricting unit includes a groove portion provided in an upper surface, and
the roller portion is inserted into the groove portion.

8. The treatment facility according to claim 1, further comprising:
a treatment table on which the irradiation target is placed; and
a control unit that performs calculations related to the treatment,
wherein the control unit calculates a direction and a position of the treatment table based on a treatment plan to determine a sliding direction of the movable door.

9. The treatment facility according to claim 8,
wherein the control unit controls the movable door such that the movable door slides in the determined sliding direction.

10. The treatment facility according to claim 8, further comprising:
a storage unit that stores various types of data necessary for the control unit to perform the calculations related to the treatment.

11. The treatment facility according to claim 1, further comprising:
a detection unit that detects a position of the movable door,
wherein the detection unit is provided on a ceiling side.

12. The treatment facility according to claim 11,
wherein a plurality of the detection units are provided, and
the plurality of detection units include a detection unit that detects that the movable door is disposed at a position for covering the irradiation port and a detection unit that detects that the movable door is disposed at a retracted position.

13. The treatment facility according to claim 1, further comprising:
a treatment room in which the irradiation target is treated; and
an accelerator room in which an accelerator is disposed.

14. The treatment facility according to claim 13,
wherein the treatment room and the accelerator room are separated by the wall portion.

15. The treatment facility according to claim 1,
wherein the radiation is a neutron ray.

16. A radiation treatment apparatus that irradiates an irradiation target with radiation to perform treatment, the radiation treatment apparatus comprising:
an irradiation unit that emits the radiation; and
a movable door that is provided to face a wall portion, in which an irradiation port of the irradiation unit is provided, so as to cover the irradiation port,
wherein the movable door slides along the wall portion to both sides of the irradiation port in a lateral direction.
